(19) United States
(12) Patent Application Publication
(10) Pub. No.: US 2023/0127826 A1
(43) Pub. Date: Apr. 27, 2023

(54) CATHETER WITH MULTIPLE BRAID LAYERS

(71) Applicant: Surmodics MD, LLC, Eden Prairie, MN (US)

(72) Inventors: Derek Stratton, Bloomington, MN (US); Karl Ganske, Edina, MN (US); Mike Kuvaas, Hutchinson, MN (US); Jacob Chmielewski, Clear Lake, MN (US)

(21) Appl. No.: 17/961,849

(22) Filed: Oct. 7, 2022

Related U.S. Application Data
(60) Provisional application No. 63/253,760, filed on Oct. 8, 2021.

(51) Int. Cl. A61M 25/00

(52) U.S. Cl. CPC A61M 25/005; A61M 25/0013; A61M 25/001; A61M 2025/0062

(57) ABSTRACT

Embodiments herein relate to catheters including multiple braid layers. In an embodiment, a catheter is included having a catheter body, the catheter body can include a proximal end portion; a distal end portion; an inner liner; and a braided structure, the braided structure can include an inner braid layer, the inner braid layer can include a first set of inner wires; and a second set of inner wires; an outer braid layer, the outer braid layer can include a first set of outer wires; and a second set of outer wires; wherein the braided structure is disposed over the inner liner; wherein the first set of inner wires are larger than the second set of inner wires; and wherein the first set of outer wires are larger than the second set of outer wires. Other embodiments are also included herein.

This application claims the benefit of U.S. Provisional Application No. 63/253,760, filed Oct. 8, 2021, the content of which is herein incorporated by reference in its entirety.

### FIELD

Embodiments herein relate to catheters including multiple braid layers.

### BACKGROUND

Catheters have many applications in the context of medical treatment. Microcatheters are those with a relatively small diameter that make them ideal for navigating complex vasculature within the human body.

Desirable properties of catheters can include pushing capabilities (sometimes referred to as “pushability”) such that force provided by a clinician to the proximal end of the catheter can be transmitted sufficiently through the catheter to advance it to a particular site. Desirable properties of catheters can also include torque transmission properties, such that torque applied to the proximal end of the catheter can be transmitted to the distal end.

### SUMMARY

Embodiments herein relate to catheters including multiple braid layers. In a first aspect, a catheter can be included having a catheter body. The catheter body can include a proximal end portion, a distal end portion, an inner liner, and a braided structure. The braided structure can include an inner braid layer. The inner braid layer can include a first set of inner wires and a second set of inner wires. The braided structure can include an outer braid layer. The outer braid layer can include a first set of outer wires and a second set of outer wires. The braided structure can be disposed over the inner liner. The first set of inner wires can be larger than the second set of inner wires and the first set of outer wires can be larger than the second set of outer wires.

In a second aspect, in addition to one or more of the preceding or following aspects, or in the alternative to some aspects, the first set of inner wires wrap around the inner liner in a direction that can be opposite to a direction that the first set of outer wires wrap around the inner liner.

In a third aspect, in addition to one or more of the preceding or following aspects, or in the alternative to some aspects, the outer braid layer extends farther toward the distal end portion than the inner braid layer.

In a fourth aspect, in addition to one or more of the preceding or following aspects, or in the alternative to some aspects, the outer braid layer extends farther toward the distal end portion than the inner braid layer by 5 to 8 millimeters.

In a fifth aspect, in addition to one or more of the preceding or following aspects, or in the alternative to some aspects, the catheter body can include a jacket layer, wherein the jacket layer can be disposed over and penetrates into the braided structure.

In a sixth aspect, in addition to one or more of the preceding or following aspects, or in the alternative to some aspects, the jacket layer can include a thermoplastic polymer.

In a seventh aspect, in addition to one or more of the preceding or following aspects, or in the alternative to some aspects, the jacket layer can include reflowed PEBAX.

In an eighth aspect, in addition to one or more of the preceding or following aspects, or in the alternative to some aspects, the jacket layer can include a proximal jacket portion, a distal jacket portion, and a tip portion. The tip portion can be formed of a polymer having a different Shore hardness than a polymer forming the distal jacket portion.

In a ninth aspect, in addition to one or more of the preceding or following aspects, or in the alternative to some aspects, the catheter body can include a lubricious outer coating, wherein the lubricious outer coating can be disposed over an outside of the catheter body.

In a tenth aspect, in addition to one or more of the preceding or following aspects, or in the alternative to some aspects, the lubricious outer coating can include a hydrophilic polymer.

In an eleventh aspect, in addition to one or more of the preceding or following aspects, or in the alternative to some aspects, the inner liner can define a lumen, wherein the lumen can be configured to receive a guidewire.

In a twelfth aspect, in addition to one or more of the preceding or following aspects, or in the alternative to some aspects, the inner liner can define a lumen, wherein the lumen can be configured to receive a guidewire having a diameter from 0.014 to 0.035 inches.

In a thirteenth aspect, in addition to one or more of the preceding or following aspects, or in the alternative to some aspects, the inner liner can include a hydrophobic polymer.

In a fourteenth aspect, in addition to one or more of the preceding or following aspects, or in the alternative to some aspects, the inner liner can include polytetrafluoroethylene.

In a fifteenth aspect, in addition to one or more of the preceding or following aspects, or in the alternative to some aspects, the inner braid layer can have a braid angle that can be less than the outer braid layer.

In a sixteenth aspect, in addition to one or more of the preceding or following aspects, or in the alternative to some aspects, the inner braid layer can have a braid angle from 25 to 45 degrees and the outer braid layer can have a braid angle from 45 to 65 degrees.

In a seventeenth aspect, in addition to one or more of the preceding or following aspects, or in the alternative to some aspects, the inner braid layer can have a braid angle from 30 to 40 degrees and the outer braid layer can have a braid angle from 50 to 60 degrees.

In an eighteenth aspect, in addition to one or more of the preceding or following aspects, or in the alternative to some aspects, the inner braid layer can have a braid angle that can be greater than the outer braid layer.

In a nineteenth aspect, in addition to one or more of the preceding or following aspects, or in the alternative to some aspects, the inner braid layer can have a braid angle from 45 to 65 degrees and the outer braid layer can have a braid angle from 25 to 45 degrees.

In a twentieth aspect, in addition to one or more of the preceding or following aspects, or in the alternative to some aspects, the inner braid layer can have a braid angle from 45 to 65 degrees and the outer braid layer can have a braid angle from 30 to 40 degrees.

In a twenty-first aspect, in addition to one or more of the preceding or following aspects, or in the alternative to some aspects, the first set of inner wires and the second set of inner wires can be at least partially flat wires.

In a twenty-second aspect, in addition to one or more of the preceding or following aspects, or in the alternative to some aspects, the first set of inner wires and the second set of inner wires can be at least partially flat stainless steel wires.

In a twenty-third aspect, in addition to one or more of the preceding or following aspects, or in the alternative to some aspects, the first set of inner wires and the second set of inner wires each include from 6 to 10 wires.

In a twenty-fourth aspect, in addition to one or more of the preceding or following aspects, or in the alternative to some aspects, the first set of inner wires and the second set of inner wires can each include 8 wires.

In a twenty-fifth aspect, in addition to one or more of the preceding or following aspects, or in the alternative to some aspects, the first set of outer wires and the second set of outer wires can be at least partially flat wires.

In a twenty-sixth aspect, in addition to one or more of the preceding or following aspects, or in the alternative to some aspects, the first set of outer wires and the second set of outer wires can be at least partially flat stainless steel wires.

In a twenty-seventh aspect, in addition to one or more of the preceding or following aspects, or in the alternative to some aspects, the first set of outer wires and the second set of outer wires each include from 6 to 10 wires.

In a twenty-eighth aspect, in addition to one or more of the preceding or following aspects, or in the alternative to some aspects, the first set of outer wires and the second set of outer wires each include 8 wires.

In a twenty-ninth aspect, in addition to one or more of the preceding or following aspects, or in the alternative to some aspects, the catheter further can include a flexible strain relief device, wherein the flexible strain relief device can be disposed over the proximal end portion of the catheter body.

In a thirtieth aspect, in addition to one or more of the preceding or following aspects, or in the alternative to some aspects, the catheter further can include a hub, wherein the hub engages a flexible strain relief device.

In a thirty-first aspect, in addition to one or more of the preceding or following aspects, or in the alternative to some aspects, the distal end portion can define a tapered tip.

In a thirty-second aspect, in addition to one or more of the preceding or following aspects, or in the alternative to some aspects, the catheter body can include a metal marker band, wherein the metal marker band can be disposed within the tapered tip.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a catheter in accordance with various embodiments herein.

FIG. 2 is a schematic view of a distal end portion in accordance with various embodiments herein.

FIG. 3 is a cross-section view of a distal end portion as taken along line 3-3′ of FIG. 2 in accordance with various embodiments herein.

FIG. 4 is a schematic view of a braided structure in accordance with various embodiments herein.

FIG. 5 is a cross-sectional view of an inner braid layer in accordance with various embodiments herein.

FIG. 6 is a schematic view of wire directions in accordance with various embodiments herein.

FIG. 7 is a cross-sectional view of an inner braid layer and an outer braid layer in accordance with various embodiments herein.

FIG. 8 is a cross-sectional view of a catheter in accordance with various embodiments herein.

FIG. 9 is a schematic view of a jacket layer in accordance with various embodiments herein.

FIG. 10 is a schematic view of a catheter in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, desirable properties of catheters can include pushability as well as torque transmission. Such properties can be challenging to provide in the context of small diameter microcatheters. However, embodiments herein include catheters, and specifically microcatheters, that have excellent pushability and torque transmission properties while also exhibiting a significant degree of resistance to kinking.

In an embodiment here, a catheter is included having a catheter body. The catheter body can include a braided structure including an inner braid layer and an outer braid layer. The inner braid layer can include a first set of inner wires and a second set of inner wires. The outer braid layer can include a first set of outer wires and a second set of outer wires. The first set of inner wires can be larger than the second set of inner wires. Further, the first set of outer wires can be larger than the second set of outer wires.

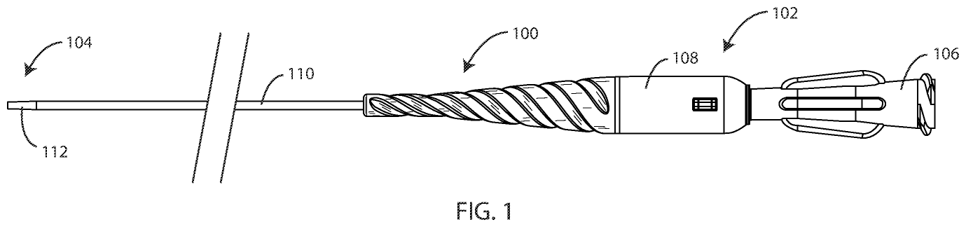

Referring now to FIG. 1, a schematic view of a catheter 100 is shown in accordance with various embodiments herein. The catheter 100 is shown with a hub 106 and a flexible strain relief device 108. The hub 106 can serve as a point of attachment or engagement with other components such as a cap structure or other components. The hub 106 can be formed of various materials. In some embodiments, the hub 106 can be formed of a thermoplastic or thermoset polymers. As a specific example, in some embodiments, the hub 106 can be formed of a polymer such as a polycarbonate. In some embodiments, the hub 106 can be substantially rigid. The hub 106 can engage the flexible strain relief device 108 directly or indirectly. In some embodiments, the hub 106 can directly engage the flexible strain relief device 108 using a snap fit connection mechanism.

The flexible strain relief device 108 can serve to prevent the formation of kinks in the catheter due to the application of force by the clinician. The flexible strain relief device 108 can take on various shapes and configurations and can be formed of various materials including thermoplastic and thermoset polymers. In some embodiments, the flexible strain relief device can be formed of an elastomeric polymer material. The flexible strain relief device can be formed of a polymer that is flexible and relatively soft. As a specific example, in some embodiments the flexible strain relief device can be formed of a polyether block amide polymer (PEBA or PEBAX).

The catheter 100 also includes a catheter body 110. The catheter body 110 includes a proximal end portion 102 and a distal end portion 104. The distal end portion 104 can define a tapered tip 112. The flexible strain relief device 108 can be disposed at least partially over the proximal end portion 102 of the catheter body 110.

As will be described more fully herein, the catheter 100 can include a braided structure disposed over an inner liner, the braided structure can include an inner braid layer and an outer braid layer. As described below, both the inner braid layer and the outer braid layer can have asymmetric torque transmission properties with respect to the direction (clockwise or counterclockwise) of applied torque.

The length of the catheter body 110 can vary. In some embodiments, the catheter body 110 length can be about 30, 40, 50, 60, 70, 80, 90, 100, 120, 140, 160, 180, 200, 220 centimeters, or longer. In some embodiments, the catheter body 110 length can fall within a range between any of the foregoing. In some embodiments, the catheter body 110 length can be about 65, 90, 135, 150, or 200 centimeters. The outer diameter of the catheter body 110 can vary. In some embodiments, the outer diameter of the catheter body 110 can be about 1.6 F (French), 1.8 F, 2.0 F, 2.2 F, 2.4 F, 2.6 F, 2.8 F, 3.0 F, 3.5 F, 4.0 F, 4.5 F, 5.0 F, 5.5 F, 6.0 F, 6.5 F, 7.0 F, 7.5 F or larger, or an outer diameter falling within a range between any of the foregoing.

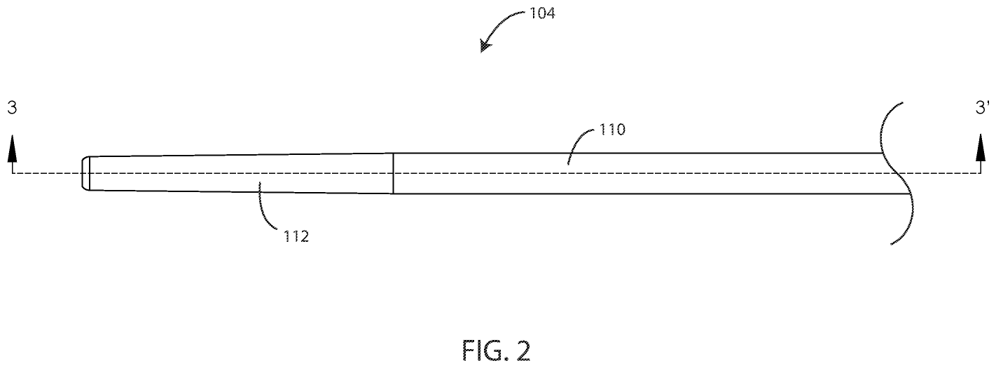

Referring now to FIG. 2, a schematic view of a distal end portion 104 of a catheter is shown in accordance with various embodiments herein. As before, the catheter includes a catheter body 110. The catheter body 110 includes the distal end portion 104 that, in turn, defines a tapered tip 112. As before, a braided structure (not visible in FIG. 2) can include an inner braid layer and an outer braid layer. As will be described further below, in various embodiments, the inner braid layer extends farther toward the distal end portion 104 than the outer braid layer.

In some embodiments, the beginning of the tapered tip 112 can coincide with where the inner braid layer extends outward beyond the end of the outer braid layer. In some embodiments, the tapered tip 112 can have a length of less than about 10, 9, 8, 7, 6, 5, 4, 3, 2, or 1 millimeters, or an amount falling within a range between any of the foregoing.

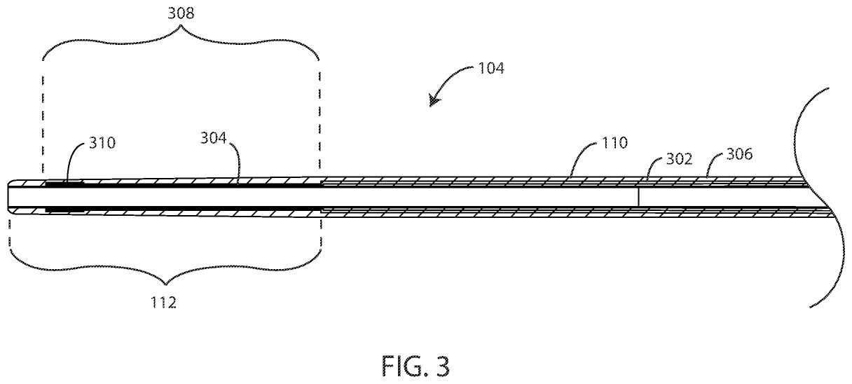

Referring now to FIG. 3, a cross-section view of a distal end portion 104 as taken along line 3-3′ of FIG. 2 is shown in accordance with various embodiments herein. As previously described, the catheter includes a catheter body 110 with a distal end portion 104 defining a tapered tip 112.

The catheter body 110 includes a braided structure including an outer braid layer 302 and an inner braid layer 304. The inner braid layer 304 extends farther toward the distal end portion 104 than the outer braid layer 302 by a distance 308. The distance 308 can be 3, 4, 5, 6, 7, 8, 9, 10, 11, 12 millimeters or more, or an amount falling within a range between any of the foregoing. In various embodiments, the inner braid layer 304 extends farther toward a distal end portion 104 than an outer braid layer 302 by 5 to 8 millimeters. However, in various embodiments, the outer braid layer 302 extends farther toward a distal end portion 104 than an inner braid layer 304. For example, in various embodiments, the outer braid layer 302 extends farther toward a distal end portion 104 than an inner braid layer 304 by 5 to 8 millimeters.

The catheter body 110 also includes a jacket layer 306. In various embodiments, the jacket layer 306 can be disposed over and/or penetrate into the braided structure. In some embodiments, the jacket layer 306 can be an extrusion. After placement of the jacket layer 306 over the braided structure, the jacket layer 306 can be reflowed such that it is disposed over the braided structure as well as at least partially within interstices of the braided structure. In some embodiments, after reflowing, the jacket layer 306 can be disposed over the braided structure as well as at least partially within interstices of both the inner braid layer 304 and the outer braid layer 302 of the braided structure.

In this specific example, the catheter body 110 also includes a metal marker band 310. In various embodiments, the metal marker band 310 can be disposed within a tapered tip. Various metals can be used to form the metal marker band 310. In some embodiments, the metal marker band 310 can be formed of a platinum iridium alloy. In some embodiments, the metal marker band 310 can be disposed over a distal end of the inner braid layer 304. In some embodiments, the metal marker band 310 can be crimped in place. In some embodiments, the metal marker band 310 can be swaged down onto the outer braid layer 302, such as swaged down onto an end of the outer braid layer 302.

In various embodiments, the inner braid layer 304 has a braid angle that is less than an outer braid layer 302. In various embodiments, the inner braid layer 304 has a braid angle from 25 to 45 degrees and an outer braid layer 302 has a braid angle from 45 to 65 degrees. In various embodiments, the inner braid layer 304 has a braid angle from 30 to 40 degrees and an outer braid layer 302 has a braid angle from 50 to 60 degrees.

In various embodiments, the inner braid layer 304 has a braid angle that is greater than an outer braid layer 302. In various embodiments, the inner braid layer 304 has a braid angle from 45 to 65 degrees and an outer braid layer 302 has a braid angle from 25 to 45 degrees. In various embodiments, the inner braid layer 304 has a braid angle from 45 to 65 degrees and an outer braid layer 302 has a braid angle from 30 to 40 degrees. In various embodiments, the inner braid layer 304 has a braid angle of about 55 degrees and an outer braid layer 302 has a braid angle of about 35 degrees.

In various embodiments, the inner braid layer 304 can have a higher pick count (which can be expressed as picks per inch or PPI which refers to the number times the wire crosses for every inch of shaft length) than the outer braid layer 302. For example, in various embodiments, the inner braid layer 304 can have a braid with 120 to 180 PPI (or 145 to 155 PPI) and the outer braid layer 302 can have a braid with 45 to 85 PPI (or 55 to 75 PPI). In various embodiments, the inner braid layer 304 can have a braid with about 150 PPI and the outer braid layer 302 can have a braid with about 65 PPI. However, in some embodiments, the inner braid layer 304 can have a lower pick count or PPI than the outer braid layer 302.

In some embodiments, the pick count or pic count can be consistent along the length of the catheter for the inner braid layer 304, the outer braid layer 302, or both. However, in some embodiments, the pick count can vary along the length of the catheter for the inner braid layer 304, the outer braid layer 302, or both. Varying the pick count along the length of the catheter in either layer is a technique that can be used herein to provide optimal characteristics at different points along the length of the catheter. Generally speaking, all things being equal, higher torque can be achieved with a higher pick count, but this can reduce the flexibility of the resulting shaft. While lower pick count tends to orient the wires more in parallel to the longitudinal axis of the catheter, which improves pushability and flexibility. As such, varying the pick count can provide different functional properties at different points along the length of the catheter. In some embodiments, the inner braid layer 304 and/or the outer braid layer 302 can include a first portion and a second portion wherein the pick count or PPI varies between the two portions by at least 5, 10, 15, 20, 25, 30, 40, 50, 75, 100, 200 percent or more, or an amount falling within a range between any of the foregoing.

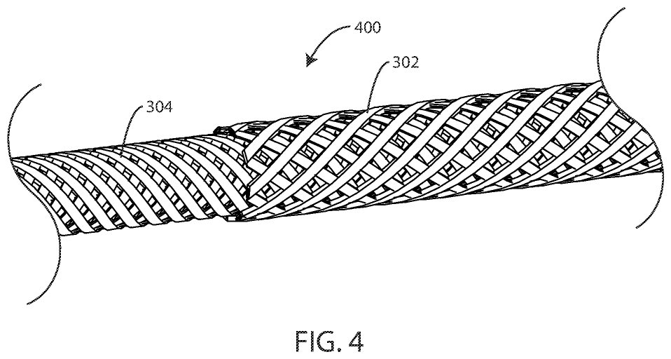

Referring now to FIG. 4, a schematic view of a braided structure 400 is shown in accordance with various embodiments herein. As before, the braided structure 400 includes an outer braid layer 302. The braided structure 400 also includes an inner braid layer 304. In this example, the outer braid layer 302 terminates and the inner braid layer 304 extends outward beyond the end of the outer braid layer 302. However, in various embodiments, the outer braid layer can overlap the ends of the inner braid layer. For example, the outer braid layer can overlap a proximal end of the inner braid layer and/or overlap a distal end of the inner braid layer. As such, in some embodiments, the outer braid layer can extend along a greater length of the catheter than the inner braid layer. In other embodiments the inner braid layer and the outer braid layer can be substantially coterminous.

In some embodiments, one or both of the outer braid layer 302 and the inner braid layer 304 can include welded terminations. In some embodiments, the terminations of the outer braid layer 302 and the inner braid layer 304 can be etched or electropolished.

The ends of a braid layer can be secured to the other components of the catheter in various ways including using various types of fastening elements, adhesives, other structures, or the like. In some embodiments, a layer of a heat shrink tubing (including, but not limited to a PET heat shrink tubing material or another type of polymeric heat shrink tubing) can be disposed over an end (proximal end, distal end, or both) of a braid layer in order to secure it down to other catheter components.

Various braid patterns can be utilized herein. In some embodiments, the braid pattern can be a one wire under two wires, then over two wires braid pattern (sometimes referred to as a full load braid pattern) or one over two, under two. Such a braid pattern can be used for the inner braid layer 304 and/or the outer braid layer 302. However, other braid patterns can also be used herein. For example, in some embodiments, a diamond braid pattern, a one over one pattern, a chase wire pattern, or the like could be used. In some embodiments, an inner braid layer can have one type of braid pattern while the outer braid layer can have another type of braid pattern. However, in some embodiments, both the inner braid layer and the outer braid layer can have the same braid pattern. In various embodiments the braid can have a right hand lay. In various embodiments, the braid can have a left hand lay. In some embodiments, an inner braid layer herein can have one type of lay while the outer braid layer can have another type of lay. For example, in some embodiments, the inner braid layer can have a right hand lay while the outer braid layer can have a left hand lay. Various systems can be used to form braid structures described herein including braiding systems commercially available from Steeger USA, Inman, S.C. and Kyoritsu Co. Ltd., as well as others.

In some embodiments, catheters herein can specifically lack a coil pattern arrangement of wires. Coil pattern arrangements of wires can offer kink resistance, but may lack sufficient pushability. Embodiments of catheters herein can offer desirable levels of kink resistance even without a coil. However, in other embodiments, a coil pattern of wires can also be included.

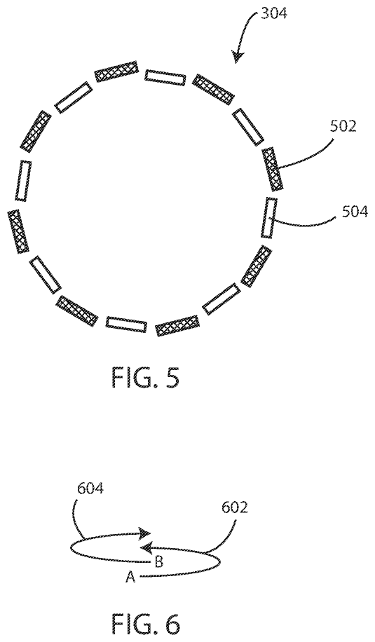

Referring now to FIG. 5, a cross-sectional view of an inner braid layer 304 is shown in accordance with various embodiments herein. The inner braid layer 304 includes a first set of inner wires 502. The inner braid layer 304 also includes a second set of inner wires 504. The first set of inner wires 502 and the second set of inner wires 504 can have various shapes in cross-section and can be the same or different from one another. In some embodiments, the cross-sectional shape can be circular, square, rectangular, ovoid, polygonal, of the like. In various embodiments, the first set of inner wires 502 and a second set of inner wires 504 are at least partially flat wires. In various embodiments, the first set of inner wires 502 and a second set of inner wires 504 are at least partially flat metal wires. Various metals can be used including, but not limited to, pure elemental metals and alloys such as stainless steel, nitinol, and the like. In various embodiments, the first set of inner wires 502 and a second set of inner wires 504 are at least partially flat stainless-steel wires.

The wires of the first set of inner wires 502 and the wires of the second set of inner wires 504 can be different in size, shape, and/or material from one another. For example, in some embodiments, the first set of inner wires 502 can be larger than the wires of the second set of inner wires 504, or vice versa. With smaller wires, generally flexibility is good but pushability and torque transmission go down. With larger wires, generally pushability and torque transmission are good, but flexibility go down. It has been found herein that optimal properties can be achieved by combining larger and smaller wires together.

In some embodiments, the larger wires (where relatively larger wires are included along with relatively smaller wires) can be flat wires having dimensions of 0.0010 to 0.0020 inches (thickness) by 0.0025 to 0.0035 inches (width) in cross-section and the smaller wires can be flat wires having dimensions of 0.0002 to 0.0008 inches by 0.0025 to 0.0035 inches in cross-section. In some embodiments, the larger wires (where relatively larger wires are included along with relatively smaller wires) can be flat wires having dimensions of approximately 0.0015 inches by 0.003 inches in cross-section and the smaller wires can be flat wires having dimensions of approximately 0.0005 inches by 0.003 inches in cross-section.

In various embodiments, the first set of inner wires 502 and a second set of inner wires 504 each comprise from 4 to 12 wires. In various embodiments, the first set of inner wires 502 and a second set of inner wires 504 each comprise from 6 to 10 wires. In various embodiments, the first set of inner wires 502 and a second set of inner wires 504 each comprise 8 wires.

In some embodiments, the first set of inner wires 502 can include a portion of wires wound in a first direction and a portion of wires wound in a second direction (such as 4 wires CW and 4 wires CCW, using 8 wires as an example) and the second set of inner wires 504 can also include a portion of wires wound in a first direction and a portion of wires wound in a second direction (such as 4 wires CW and 4 wires CCW, using 8 wires as an example). However, in other embodiments, all of the wires of the first set of inner wires 502 can be wound in one direction while all of the wires of the second set of inner wires 504 can be wound in the opposite direction. In this case, where the first set of inner wires 502 and the second set of inner wires 504 are different from one another (in size and/or material), this can result in asymmetric directional torque transmission properties provided by the braid layer.

Referring now to FIG. 6, a schematic view of wire directions is shown in accordance with various embodiments herein. FIG. 6 shows a first direction 602. FIG. 6 shows a second direction 604. In some embodiments, the first direction 602 can correspond to the direction of winding or wrapping of a first set of inner wires 502. In some embodiments, the second direction 604 can correspond to the direction of winding or wrapping of a second set of inner wires 504.

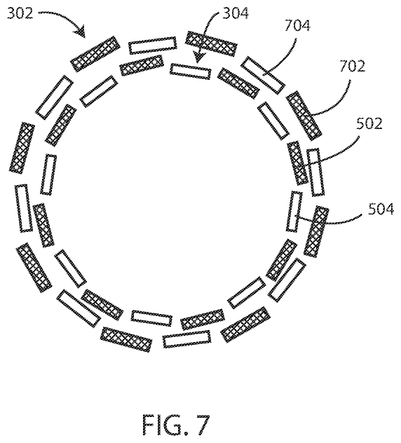

While FIG. 5 shows wires of an inner braid layer, certain properties thereof can be provided similarly within an outer braid layer. Referring now to FIG. 7, a cross-sectional view of an inner braid layer 304 and an outer braid layer 302 is shown in accordance with various embodiments herein. As before, the inner braid layer 304 includes a first set of inner wires 502 and a second set of inner wires 504.

The outer braid layer 302 includes a first set of outer wires 702. The outer braid layer 302 also includes a second set of outer wires 704. The first set of outer wires 702 and the second set of outer wires 704 can have various shapes in cross-section and can be the same or different from one another. In some embodiments, the cross-sectional shape can be circular, square, rectangular, ovoid, polygonal, of the like. In various embodiments, the first set of outer wires 702 and a second set of outer wires 704 are at least partially flat wires. In various embodiments, the first set of outer wires 702 and a second set of outer wires 704 are at least partially flat metal wires. Various metals can be used including, but not limited to, pure elemental metals and alloys such as stainless steel, nitinol, and the like. In various embodiments, the first set of outer wires 702 and a second set of outer wires 704 are at least partially flat stainless-steel wires.

In various embodiments, the first set of outer wires 702 and a second set of outer wires 704 each comprise from 4 to 12 wires. In various embodiments, the first set of outer wires 702 and a second set of outer wires 704 each comprise from 6 to 10 wires. In various embodiments, the first set of outer wires 702 and a second set of outer wires 704 each comprise 8 wires.

The wires of the first set of outer wires 702 and the wires of the second set of outer wires 704 can be different in size, shape, and/or material from one another. For example, in some embodiments, the first set of outer wires 702 can be larger than the wires of the second set of outer wires 704, or vice versa. In some embodiments, the larger wires (where relatively larger wires are included along with relatively smaller wires) can be flat wires having dimensions of 0.0010 to 0.0020 inches (thickness) by 0.0025 to 0.0035 inches (width) in cross-section and the smaller wires can be flat wires having dimensions of 0.0002 to 0.0008 inches by 0.0025 to 0.0035 inches in cross-section. In some embodiments, the larger wires (where relatively larger wires are included along with relatively smaller wires) can be flat wires having dimensions of approximately 0.0015 inches by 0.003 inches in cross-section and the smaller wires can be flat wires having dimensions of approximately 0.0005 inches by 0.003 inches in cross-section.

In some embodiments, the first set of outer wires 702 can include a portion of wires wound in a first direction and a portion of wires wound in a second direction (such as 4 wires CW and 4 wires CCW, using 8 wires as an example) and the second set of outer wires 704 can also include a portion of wires wound in a first direction and a portion of wires wound in a second direction (such as 4 wires CW and 4 wires CCW, using 8 wires as an example). However, in other embodiments, all of the wires of the first set of outer wires 702 can be wound in one direction while all of the wires of the second set of outer wires 704 can be wound in the opposite direction. In this case, where the first set of outer wires 702 and the second set of outer wires 704 are different from one another (in size and/or material), this can result in asymmetric directional torque transmission properties provided by the braid layer. In some embodiments, asymmetric directional torque transmission properties provided by the outer braid layer 302 can be directionally different (clockwise vs. counterclockwise) than the asymmetric directional torque transmission properties provided by the inner braid layer 304.

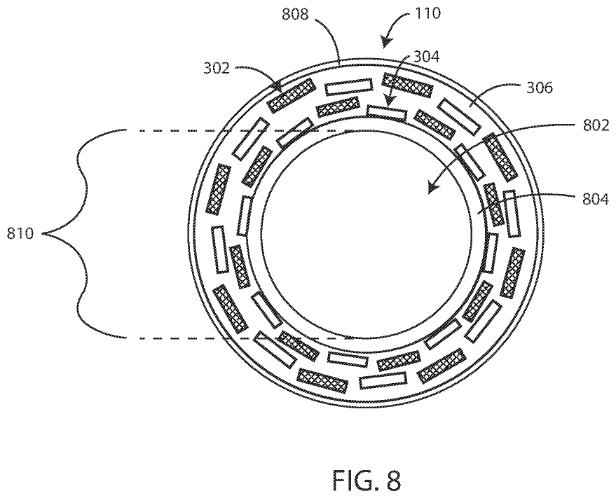

Referring now to FIG. 8, a cross-sectional view of a catheter 100 is shown in accordance with various embodiments herein. The catheter 100 includes a catheter body 110 and a braided structure therein. The braided structure includes an outer braid layer 302 and an inner braid layer 304. The catheter body 110 also includes a jacket layer 306. In this example, the jacket layer 306 is disposed over the braided structure, but also is disposed within the braided structure.

The catheter body also includes an inner liner 804. In various embodiments, the inner liner 804 can include thermoplastic or thermoset polymers. In various embodiments, the inner liner 804 can include a hydrophobic polymer. In various embodiments, the inner liner 804 can include polytetrafluoroethylene (PTFE).

The inner liner 804 defines a lumen 802 having a diameter 810. In various embodiments, the lumen 802 of the inner liner 804 can be configured to receive a guidewire. In various embodiments, the lumen 802 can be configured to receive a guidewire having a diameter from 0.014 to 0.035 inches and thus be sized to be slightly larger than the same. In some embodiments, the lumen 802 can have a consistent inner diameter all along the catheter body. However, in other embodiments the lumen 802 can transition to a tapered narrower inner diameter at the distal end portion 104 of the catheter 100.

A lubricious outer layer 808 or coating can be disposed on the catheter body 110. The lubricious outer layer 808 can include a polymer providing lubricious properties. In various embodiments, the lubricious outer layer 808 can include a hydrophilic polymer. Exemplary polymers of the lubricious outer layer 808 are described in greater detail below.

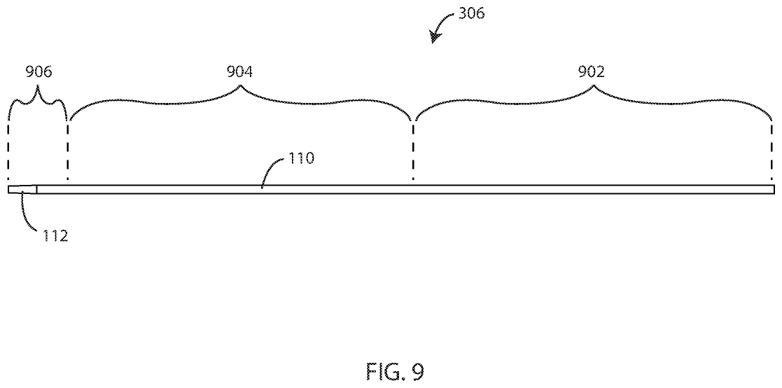

In some embodiments, the jacket layer of the catheter can have different zones or portions. Referring now to FIG. 9, a schematic view of a catheter body 110 including jacket layer 306 is shown in accordance with various embodiments herein. Jacket layer 306 can include a proximal jacket portion 902, a distal jacket portion 904, and a tip portion 906. The tip portion 906 can include the tapered tip 112 of the catheter.

In various embodiments, the jacket layer 306 can include a thermoplastic polymer. In various embodiments, the jacket layer 306 can include a thermoplastic polymer that is reflowed so that it penetrates into the braided structure of the catheter. In various embodiments, the jacket layer 306 can include a reflowed PEBAX polymer.

Various portions of the jacket layer can be formed of different materials such that different portions of the jacket layer can have different properties than other portions. For example, in various embodiments, the tip portion 906 can be formed of a polymer having a different Shore hardness than a polymer forming a distal jacket portion 904.

As an example, in some embodiments, the tip portion 906 can be formed of a polymer having a Shore hardness of 68 D to 74 D, the distal jacket portion 904 can be formed of a polymer having a Shore hardness of 61 D to 65 D, and the proximal jacket portion 902 can be formed of a polymer having a Shore hardness of 68 D to 74 D. As an example, in some embodiments, the tip portion 906 can be formed of a polymer having a Shore hardness of about 72 D, the distal jacket portion 904 can be formed of a polymer having a Shore hardness of about 63 D, and the proximal jacket portion 902 can be formed of a polymer having a Shore hardness of about 72 D.

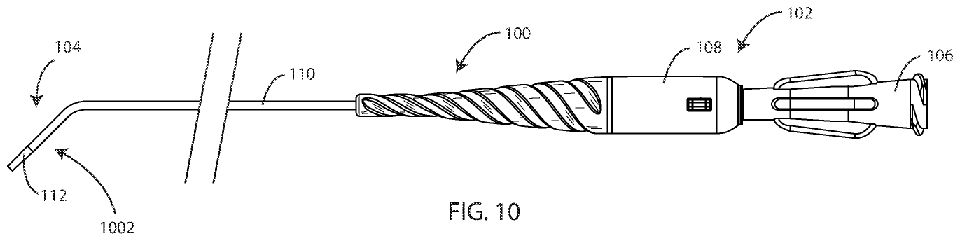

The distal end portion 104 of catheters herein can take on many different configurations. Referring now to FIG. 10, a schematic view of a catheter 100 is shown in accordance with various embodiments herein. As before, the catheter 100 includes a hub 106 and a flexible strain relief device 108. The catheter 100 also includes a catheter body 110 with a proximal end portion 102 and a distal end portion 104.

In this embodiment, the catheter body 110 includes an angled portion 1002 at the distal end portion 104 of the catheter. The angled portion 1002 can be of various sizes. In some embodiments, the angled portion 1002 can be about 2, 3, 4, 5, 6, 7, 8, 9, or 10 millimeters in length, or an amount falling within a range between any of the foregoing.

### Lubricious Outer Layer/Coating

Exemplary materials for the lubricious outer layer/coating herein can be found in U.S. Publ. Pat. App. No. 2014/0193474. In some embodiments, the lubricious polymer layer/coating includes a vinyl pyrrolidone polymer. As used herein a “vinyl pyrrolidone polymer” refers to polymers including vinyl pyrrolidone monomeric units. The vinyl pyrrolidone polymer can be a vinyl pyrrolidone homopolymer or a vinyl pyrrolidone copolymer including vinyl pyrrolidone and one or more (e.g., two, three, four, five, etc.) other monomeric units that are different than vinyl pyrrolidone. In embodiments, in a poly(vinyl pyrrolidone) copolymer, the vinyl pyrrolidone can be the primary monomer (molar quantity), such as present in an amount of greater than 50% (mol), 55% (mol) or greater, 60% (mol) or greater, 65% (mol) or greater, 70% (mol) or greater, 75% (mol) or greater, 80% (mol) or greater, 85% (mol) or greater, 90% (mol) or greater, 92.5% (mol) or greater, 95% (mol) or greater, 97.5% (mol) or 99% (mol) or greater. In exemplary embodiments, vinyl pyrrolidone is present in the copolymer in the range of about 75% (mol) to about 97.5% (mol), about 85% (mol) to about 97.5% (mol), or about 90% (mol) to about 97.5% (mol).

Other monomers that can be copolymerized with vinyl pyrrolidone to provide the vinyl pyrrolidone polymer include, but are not limited to acrylamide, methacrylamide, acrylic acid, acrylamido-2-methylpropanesulfonate (AMPS), methacrylic acid, methyl acrylate, methyl methacrylate, hydroxyethyl methacrylate, hydroxyethyl acrylate, glyceryl acrylate, glyceryl methacrylate, ethylene glycol, and derivatives of these monomers.

For example, in some embodiments, the lubricious polymer layer/coating includes a vinyl pyrrolidone polymer comprising a photoreactive group (e.g., photo-PVP). Reagents and methods for the preparation of photo-PVP can be found in references such as U.S. Pat. Nos. 4,979,959; 5,002,582; 5,263,992; 5,414,075; 5,512,329; and 5,637,460, the teaching of which are incorporated herein by reference. In some modes of practice, photo-PVP can be formed by the copolymerization of 1-vinyl-2-pyrrolidone and N-(3-aminopropyl (meth)acrylamide), which then can be derivatized with an acyl chloride (such as, for example, 4-benzoylbenzoyl chloride) under Schotten-Baumann conditions. That is, the acyl chloride reacts with the amino group of the N-(3-aminopropyl) moiety of the copolymer. An amide is formed resulting in the attachment of the aryl ketone to the polymer.

A vinyl pyrrolidone polymer comprising a photoreactive group can also be prepared by copolymerizing vinyl pyrrolidone with a monomer derivatized with a photoreactive group. Exemplary monomer derivatives include aryl ketone derivatives of hydrophilic free radically polymerizable monomers such as acrylamide, methacrylamide and AMPS. One exemplary methacrylamide-based monomer with a pendent photoreactive groups is N-[3-(4-benzoylbenzamido) propyl]methacrylamide (BBA-APMA), the synthesis which is described in Examples 1-3 of U.S. Pat. No. 5,858,653 (Duran et al.) Another exemplary methacrylamide-based monomer with a pendent photoreactive group is N-[3-(7-methyl-9-oxothioxanthene-3-carboxiamido)propyl] methacrylamide (MTA-APMA), the synthesis which is described in Examples 1-2 of U.S. Pat. No. 6,156,345 (Chudzik et al.)

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A catheter comprising:
a catheter body, the catheter body comprising
a proximal end portion;
a distal end portion;
an inner liner; and
a braided structure, the braided structure comprising
an inner braid layer, the inner braid layer comprising
a first set of inner wires; and
a second set of inner wires;
an outer braid layer, the outer braid layer comprising
a first set of outer wires; and
a second set of outer wires;
wherein the braided structure is disposed over the inner liner;
wherein the first set of inner wires are larger than the second set of inner wires; and
wherein the first set of outer wires are larger than the second set of outer wires.

2. The catheter of claim 1, wherein the first set of inner wires wrap around the inner liner in a direction that is opposite to a direction that the first set of outer wires wrap around the inner liner.

3. The catheter of claim 1, wherein the outer braid layer extends farther toward the distal end portion than the inner braid layer.

4. The catheter of claim 1, wherein the outer braid layer extends farther toward the distal end portion than the inner braid layer by 5 to 8 millimeters.

5. The catheter of claim 1, the catheter body comprising a jacket layer, wherein the jacket layer is disposed over and penetrates into the braided structure.

6. (canceled)

7. The catheter of claim 5, the jacket layer comprising reflowed PEBAX.

8. The catheter of claim 5, the jacket layer comprising:
a proximal jacket portion;
a distal jacket portion; and
a tip portion, wherein the tip portion is formed of a polymer having a different Shore hardness than a polymer forming the distal jacket portion.

9. The catheter of claim 1, the catheter body comprising a lubricious outer coating, wherein the lubricious outer coating is disposed over an outside of the catheter body.

10. The catheter of claim 1, the lubricious outer coating comprising a hydrophilic polymer.

11-14. (canceled)

15. The catheter of claim 1, wherein the inner braid layer has a braid angle that is less than the outer braid layer.

16. The catheter of claim 1, wherein the inner braid layer has a braid angle from 25 to 45 degrees and the outer braid layer has a braid angle from 45 to 65 degrees.

17. (canceled)

18. The catheter of claim 1, wherein the inner braid layer has a braid angle that is greater than the outer braid layer.

19. The catheter of claim 1, wherein the inner braid layer has a braid angle from to 65 degrees and the outer braid layer has a braid angle from 25 to 45 degrees.

20. (canceled)

21. The catheter of claim 1, wherein the first set of inner wires and the second set of inner wires are at least partially flat wires.

22. (canceled)

23. The catheter of claim 1, wherein the first set of inner wires and the second set of inner wires each comprise from 6 to 10 wires.

24. (canceled)

25. The catheter of claim 1, wherein the first set of outer wires and the second set of outer wires are at least partially flat wires.

26. (canceled)

27. The catheter of claim 1, wherein the first set of outer wires and the second set of outer wires each comprise from 6 to 10 wires.

28. (canceled)

29. The catheter of claim 1, further comprising a flexible strain relief device, wherein the flexible strain relief device is disposed over the proximal end portion of the catheter body.

30. (canceled)

31. The catheter of claim 1, the distal end portion defining a tapered tip.

32. (canceled)

33. The catheter of claim 1, the first set of inner wires having dimensions of 0.0010 to 0.0020 inches in thickness by 0.0025 to 0.0035 inches in width, the second set of inner wires having dimensions of 0.0002 to 0.0008 inches in thickness by 0.0025 to 0.0035 inches in width, the first set of outer wires having dimensions of 0.0010 to 0.0020 inches in thickness by 0.0025 to 0.0035 inches in width, the second set of outer wires having dimensions of 0.0002 to 0.0008 inches in thickness by 0.0025 to 0.0035 inches in width.
